(19) United States
(12) Patent Application Publication
(10) Pub. No.: US 2024/0257412 A1
(43) Pub. Date: Aug. 1, 2024

(54) SELF-CALIBRATING DENTAL DVT SUPPORTED BY MACHINE LEARNING

(71) Applicants: DENTSPLY SIRONA Inc., York, PA (US); SIRONA DENTAL SYSTEMS GMBH, Bensheim (DE)

(72) Inventors: Susanne MAUR, Bensheim (DE); Markus HUELSBUSCH, Burstadt (DE)

(73) Assignee: DENTSPLY SIRONA Inc., York, PA (US)

(21) Appl. No.: 18/578,554

(22) PCT Filed: Jul. 5, 2022
(86) PCT No.: PCT/EP2022/068520

§ 371 Date: Jan. 11, 2024

(30) Foreign Application Priority Data
Jul. 12, 2021 (EP) 21184982.3

(51) Int. Cl. G06T 11/00; A61B 6/00; A61B 6/51; G06T 7/60

(52) U.S. Cl. CPC G06T 11/005; G06T 7/60; G06T 11/006; G06T 11/008; A61B 6/51; A61B 6/5264; G06T 2207/10116; G06T 2207/20081; G06T 2207/30036; G06T 2211/424; G06T 2211/441

(57) ABSTRACT

Aspects relate to geometric calibration of a DVT imaging, by updating the geometric parameters used in a reconstruction method, in which the updating of the geometric parameters is supported by a first correction method based on machine learning (ML) by using the result of a first correction method as a reference for a second correction method for parameter estimation, and in which the second correction method for parameter estimation involves the measurement data of the DVT imaging, which includes the following steps: (S1) providing the measurement data of the DVT imaging and the geometric parameters; (S2) providing a first volume by applying a reconstruction method to the provided measurement data and the geometric parameters; (S3) providing a corrected volume by applying the first correction method to the first volume; (S4) providing updated geometric parameters by applying the second correction method to the measurement data and the corrected volume.

### TECHNICAL FIELD OF THE INVENTION

The present invention relates to a system and method for geometrically calibrating a DVT imaging in the dental field to compensate for motion artifacts in the reconstructed volume.

### BACKGROUND OF THE INVENTION

In the literature, there are methods for autocalibration that estimate the geometric parameters required for reconstruction or image correction, or an image correction for the reconstructed volume from a given DVT image. There are conventional methods which estimate the geometric parameters, for example, by evaluating the image quality or data fidelity in the reconstructed volume or by evaluating data consistency conditions. There are also correction methods based on machine learning (ML), which, for example, transform an artifact-bearing volume into an artifact-reduced volume. The respective advantages and disadvantages of the two types of methods are explained further below.

### Parameter Estimation Method

The advantages of conventional methods for parameter estimation are that the raw data are an important part of the processing chain, thus the raw data coverage, or data fidelity, can be checked and controlled. The changes to image data can be controlled, e.g. by introducing boundary conditions.

The disadvantages of conventional methods for parameter estimation are that they involve complex optimization problems with many parameters to be estimated, with many parameter dependencies and with many local optima, which cannot be solved efficiently. The computation is usually iterative. In practice, this leads to long computation times and mostly sub-optimal results.

### ML Correction Method

The advantages of the ML correction method are that it results in significantly shorter computation times than conventional methods for parameter estimation since the application of an ML correction method corresponds to a direct computation and thus does not contain an optimization problem. The more global view of the image data and their correlations leads to a faster and more efficient correction of large errors.

The disadvantages of ML correction methods are that image-to-image transformations can invent data. Also, changes to image data or parameters are more difficult to control because boundary conditions are more difficult to incorporate into ML correction methods.

An ML correction method known in the prior art is given below: Jiang et al, “Wasserstein generative adversarial networks for motion artifact removal in dental CT imaging,” Proc. SPIE 10948, Medical Imaging 2019: Physics of Medical Imaging, 2019.

### DISCLOSURE OF THE INVENTION

An objective of the present invention is to automatically geometrically calibrate a specific imaging, such as a patient imaging. Thus, outdated device calibrations can be updated or patient-specific adjustments, such as patient motion, can be corrected. This allows compensation for motion artifacts in the reconstructed volume.

This objective is achieved by the method according to claim 1. The subject-matters of the dependent claims relate to further developments and preferred embodiments.

The method according to the invention is for geometric calibration of a DVT imaging by updating the geometric parameters used in the reconstruction method, wherein the updating of the geometric parameters is supported by a first correction method based on machine learning (ML) by using the result of the first correction method as a reference for a second correction method for parameter estimation, and wherein the second correction method for parameter estimation involves the measurement data of the DVT imaging. The method comprises the following steps: Providing the measurement data of the DVT imaging and the geometric parameters; Providing a first volume by applying a reconstruction method to the provided measurement data and the geometric parameters; Providing a corrected volume by applying the first correction method to the first volume; Providing updated geometric parameters by applying the second correction method to the measurement data and the corrected volume.

A key feature of this invention is the combination of conventional methods for parameter estimation with ML correction methods.

An advantageous effect of the present invention is to support the method for automatic geometric calibration of dental patient imaging with data-based prior knowledge generated using ML correction method. The ML correction method generates data-based prior knowledge that supports/stabilizes/accelerates/controls the conventional method for parameter estimation. Thus, the advantages of both methods can be combined and their disadvantages minimized.

Specifically, the result of an ML correction method can be used as an initial estimate for a conventional correction method for parameter estimation. Preferably, the corrected volume is used in an iterative method as a regularization of a conventional method. Alternatively, it indicates where and how much change is expected in the data.

This allows to preserve the raw data coverage/data fidelity in the overall procedure and to integrate boundary conditions. Also, the speed advantage of the ML correction method and its more global view of the image data is used. The ML correction method implements a global view of the problem and thus steers towards the vicinity of the global optimum. The conventional method for parameter estimation, on the other hand, corresponds to a more local consideration of the problem and thus improves the accuracy and correctness/data fidelity of the result.

### BRIEF DESCRIPTION OF THE DRAWINGS

In the following description, the present invention will be explained in more detail with reference to exemplary embodiments and with reference to the drawings, wherein.

FIG. 1—shows a flow diagram according to one embodiment of the invention;

FIG. 2—shows a flow diagram according to another embodiment of the invention;

FIG. 3—shows a flow diagram according to a further embodiment of the invention;

FIG. 4—shows a flow diagram according to a further embodiment of the invention;

FIG. 5—shows a computerized DVT system on which the method according to the invention can be performed;

FIG. 6—shows the local interpolation and extrapolation of the geometric parameters in the volume.

The reference numerals shown in the drawings designate the elements listed below, which will be referred to in the following description of exemplary embodiments.
1 DVT system
2. X-ray device
3. X-ray source
4. X-ray detector
5. Control unit
6. Head fixation
7. Bite
8. Computer
9. Display
a. Skull of the patient
b Curved beam
c. First selected sub region of the volume
d. Second selected sub region of the volume

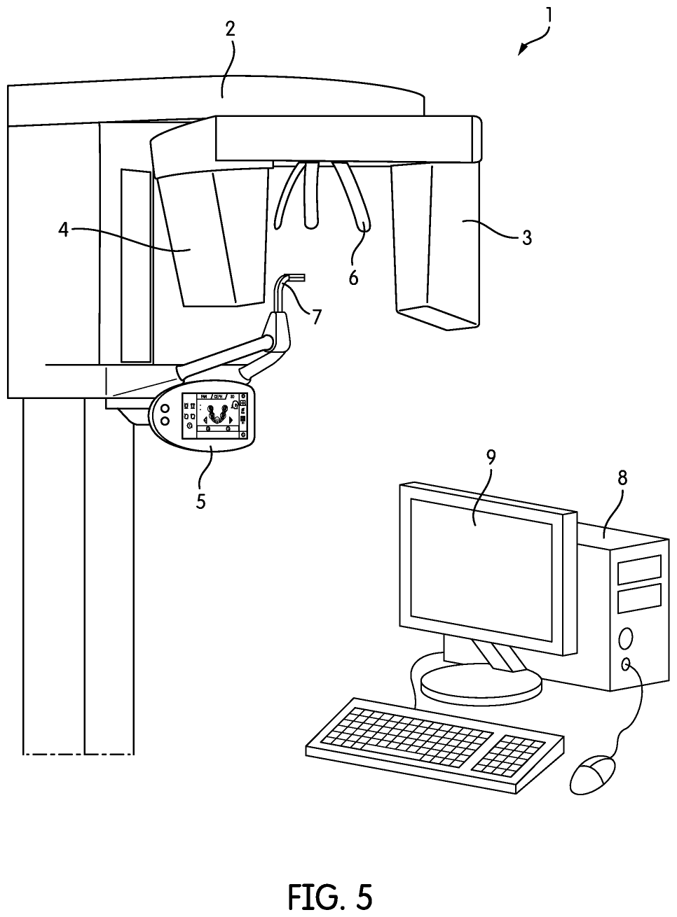

The method according to the invention is a computer implementable method, and can be executed on a computerized DVT system (1). FIG. 5 shows an example of an embodiment for a DVT system (1). In this regard, the present invention also includes a computer program having computer-readable code. The computer program may be provided on a data storage device. The computerized DVT system (1) comprises an X-ray device (2) for performing patient imaging, whereby measurement data is generated. The measurement data comprises the sinogram of the exposure consisting of X-ray projections. The X-ray device (2) has an X-ray source (3) and X-ray detector (4), which are rotated around the patent head during the exposure. The patient's head is positioned in the X-ray device with the bite (7) and the head fixation (6). The computerized DVT system (1) comprises an control unit (5), preferably a computer (8) or a computing unit connectable to the X-ray device (2), and preferably a display (9), inter alia to visualize the data sets. The computer (8) may be connected to the X-ray device (2) via a local area network (not shown) or alternatively via the Internet. The computer (8) may be part of a cloud. Alternatively, the computer (3) may be integrated into the X-ray device (2). The computations may alternatively take place in the cloud. The computer (8) executes the computer program and provides the data sets, including for visualization on the display (9). The display (9) may be spatially separated from the X-ray device (2). Preferably, the computer (8) may also control the X-ray device (2). Alternatively, separate computers may be used for the control and the reconstruction.

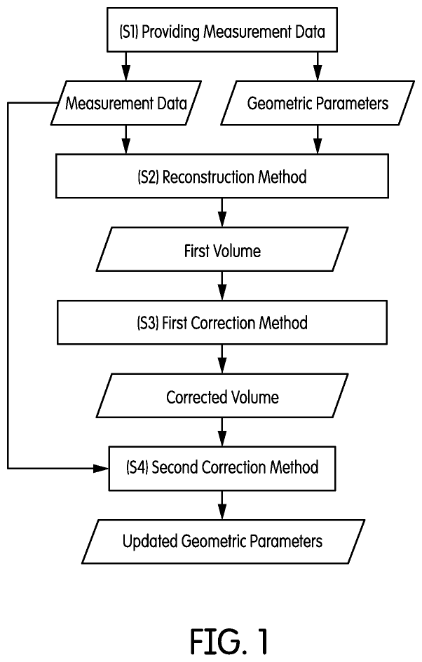

FIG. 1 shows a flow diagram according to the invention. The method according to the invention is used for geometric calibration of a DVT imaging by updating the geometric parameters used in the reconstruction method. The updating of the geometric parameters is supported by a first correction method based on machine learning (ML), by using the result 5 of the first correction method as a reference for a second correction method for parameter estimation. The second correction method for parameter estimation incorporates the measurement data of the DVT imaging.

The method comprises the steps of: (S1) providing measurement data of the DVT imaging and geometric parameters; (S2) providing a first volume by applying a reconstruction method to the provided measurement data and geometric parameters; (S3) providing a corrected volume by applying the first correction method to the first volume; (S4) providing updated geometric parameters by applying the second correction method to the measurement data and the corrected volume.

The geometric parameters or updated geometric parameters describe the projection geometry of the DVT imaging relative to the patient's head. The projection geometry preferably consists of intrinsic parameters and extrinsic parameters, wherein the intrinsic parameters comprise the relative location between the X-ray source (3) and the X-ray detector (4) as well as their resolution and the extrinsic parameters comprise a transformation consisting of rotation and translation per projection image.

The neural network (machine learning (ML)) is trained with the following data pairs:
Volume of a DVT imaging without motion artifacts and
Volumes reconstructed from the sinogram of the DVT imaging with simulated patient motion or device motion. Further alternatives will be explained later in the following description.

Data pre-processing steps on image data (sinogram or volume) may be optionally added prior to step 3 and/or step 4, such as contrast enhancement or highlighting of relevant edges or noise reduction, which align the image data so that the respective methods works better with them.

Step (3) and step (4) are applied to at least partially overlapping regions of the first or the corrected volume, wherein preferably step (3) is applied to larger regions of the first or the corrected volume than step (4).

The geometric parameters in step (S1) may preferably be calculated from the actuated and/or expected device movement. Alternatively, they may be determined by a geometric device calibration performed at an earlier time. The reconstruction method in step (S2) may be a reconstruction method according to Feldkamp, according to Davis and Kress, or an iterative reconstruction method or an algebraic reconstruction method or a statistical reconstruction method. In a preferred embodiment, the second correction method for parameter estimation from step (S4) comprises a registration method that registers the measurement data with the corrected volumes from step (S3). The registration method determines the updated geometric parameters describing the exposure-location relationship between the measurement data and the corrected volume. In one embodiment, the registration of the sinogram is performed by generating simulated sinograms using the geometric parameters and comparing them to the sinogram using similarity measures. The similarity measure is maximized by varying the geometric parameters. In generating a simulated sinogram, a forward projection of the corrected volume is performed using the geometric parameters. When the correct geometric parameters are applied, the similarity between the simulated sinogram and the sinogram is high. Since the corrected volume already contains a correction for motion artifacts, the second correction method calculates the appropriate geometric parameters for this correction. The knowledge about the geometric parameters allows subsequent correction steps or a reconstruction considering raw data coverage or data fidelity.

In an alternative embodiment, the second correction method for parameter estimation from step (S4) comprises an iterative reconstruction method that uses the corrected volume from step (S3) for regularization. An iterative reconstruction method varies any reconstruction parameters, such as the geometric parameters, and iteratively improves the raw data coverage of the reconstruction. A regularization includes the difference of the corrected volume to the reconstructed volume as a penalty term. The use of regularization stabilizes or accelerates the underlying method for parameter optimization.

In a further preferred embodiment, the second correction method for parameter estimation of step (S4) is performed only on one or more selected subregions of the corrected volume and/or one or more selected subregions of the measurement data. Sub-regions of the corrected volume may also be individual volume layers. The selection takes place by comparing the corrected volume and the first volume, or alternatively takes place directly by the first correction method, or alternatively takes place by manual selection. Executing the second correction method on selected sub-regions of the corrected volume and/or a selected sub-region of the measurement data offers the advantages of reducing the computing time and resource consumption of the second correction method. Moreover, non-rigid motions can thus be locally approximated by rigid motions. The selection of a sub-region of the corrected volume can be performed by comparison with the first volume, by identifying regions with significant changes for the correction. The selection of sub-regions of the measured data may be performed by temporal subsampling, temporal selection of the projection images or by local selection in the projection images. For this purpose, the selected subregions of the corrected volume can be projected forward into the sinogram, taking into account the geometric parameters. The alternative selection of the sub regions by the first correction method has the advantage that the ML method can determine the significance of the changes in the volume independently of the difference of the volumes. Also, the temporal selection of the projection images by evaluating the direction of motion and the time of motion can be easily realized within the first correction method.

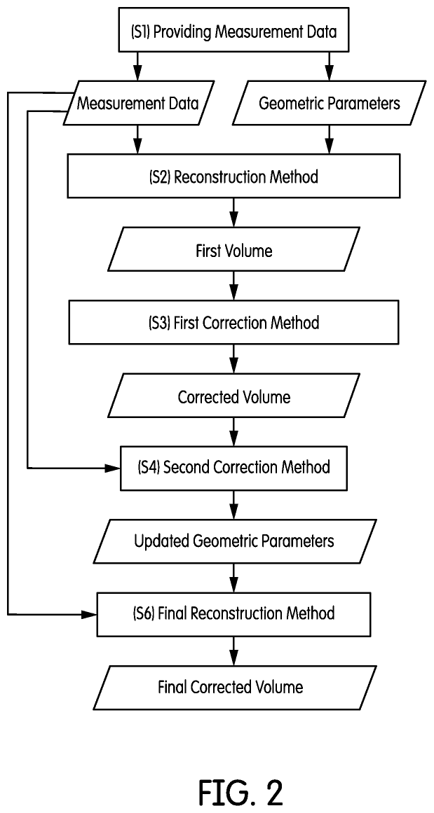

FIG. 2 shows another preferred embodiment in which the method comprises a further step (S6) for providing a final corrected volume by applying a final reconstruction method to the measurement data and the updated geometric parameters from step (S4). This step (S6) generates a volume with corrected motion artifacts taking into account the raw data coverage.

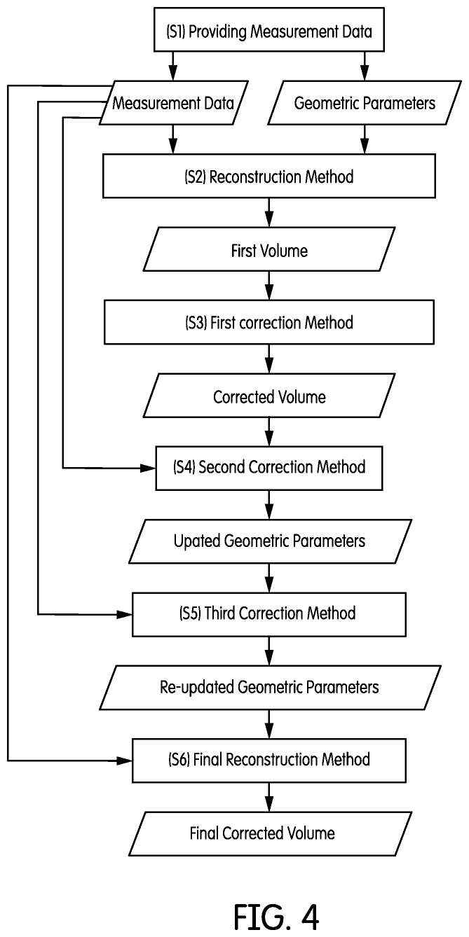

FIG. 4 shows another alternative preferred embodiment. In this embodiment, the method comprises a step (S5) for providing re-updated geometric parameters by applying a third correction method for parameter estimation to the measurement data and the updated geometric parameters from step (S4). And the method comprises an alternative step (S6′) for providing a final corrected volume by applying a final reconstruction method to the measurement data and the re-updated geometric parameters from step (S5). Step (S5) allows a further correction method for parameter estimation to be used to again improve the geometric parameters, thereby improving the initial estimate of the first correction method. In one embodiment, this may comprise the following steps, which are preferably iteratively repeated until a convergence criterion is reached: reconstruction of a temporary volume with estimated projection geometry and estimation of the projection geometry by registration of the projection images of the sinogram with the temporary volume. In one embodiment, registration of the sinogram is performed by generating simulated sinograms using the geometric parameters and comparing them to the sinogram using similarity measures. The similarity measure is maximized by varying the geometric parameters. In generating a simulated sinogram, a forward projection of the corrected volume is performed using the geometric parameters. Step (S6′) generates a final corrected volume with corrected motion artifacts considering the raw data coverage.

In addition to the image data, initial geometric parameters (e.g. data of the current device calibration) can also be transferred as input data to the method steps (3)-(5).

In further preferred alternative embodiments, sub-regions of the final volume are generated in the final reconstruction method of step (S6;S6′), and these are combined to form the final volume. If the correction methods in step (S4) and/or step (S5) are performed on a plurality of sub-regions of the volume or measurement data, thereby a set of geometric parameters is determined for each sub-region. Each set of geometric parameters may be used to reconstruct a sub-region of the volume. The sub-regions of the volume can be merged in the final volume. If the sub regions of the volume overlap, they can be suitably blended or combined.

If the correction methods in step (4) and/or step (5) are performed on one or more selected sub-regions of the volume or of the measurement data, thereby a set of geometric parameters is determined per selected sub-region. The sets of geometric parameters may be interpolated or extrapolated to determine geometric parameters for the non-selected sub-regions of the volume or of the measurement data. Here, the subregions in the volume can be selected and interpolated or extrapolated in the measurement data and vice versa.

In further preferred alternative embodiments, the final reconstruction method of step (S6;S6′) interpolates or extrapolates the updated geometric parameters of the sub-regions of step (S4) or the re-updated geometric parameters of the sub-regions of step (S5), wherein the interpolation or extrapolation weights are determined from the relative location of the non-selected sub-regions in the volume to the selected sub-regions in the volume.

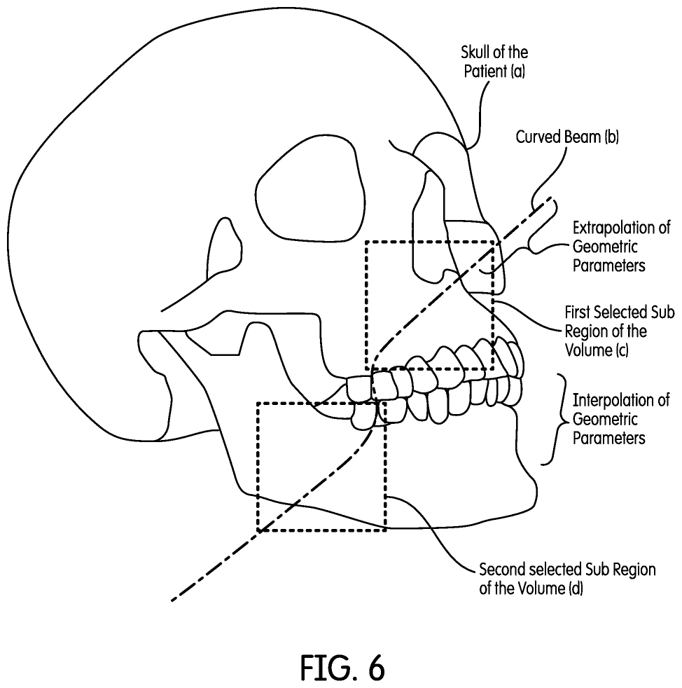

In one embodiment with multiple selected sub-regions, the geometric parameters may be defined locally in the volume. Thus, geometric parameters can be determined per voxel, so that a transformation is described per voxel and projection image. This allows the realization of non-straight or curved rays in the reconstruction, so that non-rigid motions can be considered in the reconstruction. The curved rays can be understood as each being a composition of straight sections resulting from the movement or deformation of the patient anatomy during the imaging. FIG. 6 shows a curved beam (b) through the patient's skull (a), which represents the path of the X-rays during the imaging for a rigid volume in the reconstruction. The geometric parameters are interpolated for the reconstruction between the two selected sub-regions of the volume (c,d) and extrapolated outside the two selected sub-regions of the volume (c,d).

In further alternative preferred embodiments, in the final reconstruction method from step (S6;S6′), the updated geometric parameters of the sub-regions from step (S4) or the re-updated geometric parameters of the sub-regions from step (S5) are interpolated or extrapolated in the measurement data, wherein the interpolation or extrapolation weights are determined from the relative spatial or temporal location of the non-selected sub-regions of the measurement data to the selected sub-regions of the measurement data.

In one embodiment, the geometric parameters may be temporally interpolated or extrapolated in the measurement data by interpolating or extrapolating the geometric parameters in the temporal dimension of the sinogram. In a further embodiment, the geometric parameters may be interpolated or extrapolated locally in the measurement data by interpolating or extrapolating the geometric parameters in the spatial dimensions of the sinogram.

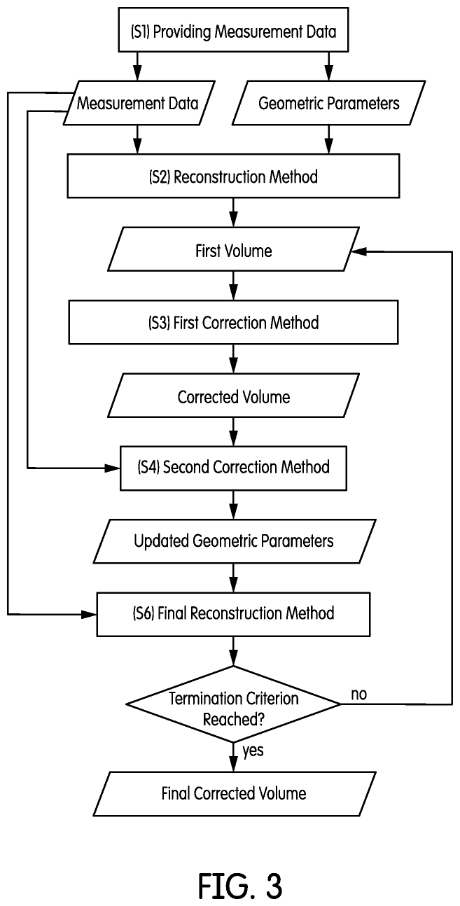

FIG. 3 shows another alternative preferred embodiment. In this embodiment, steps (S3), (S4) and (S6) are repeated one or more times or are also performed iteratively. The iterative repetition of said steps increases the correctness, accuracy and/or convergence of the correction methods, since the correction methods generally lead to a better final result for input data with a small error than for input data with a large error. The iterative repetition is terminated when a convergence criterion is reached. Possible convergence criteria are: a) whether the change in projection geometry is less than a threshold; b) whether the change in the final volume is less than a threshold; c) whether the number of iteration steps is greater than a threshold; d) whether the computation time is greater than a threshold.

In further preferred alternative embodiments, steps (S3) to (S6) are repeated one or more times or are also performed iteratively. The iterative repetition of said steps increases the correctness, accuracy and/or convergence of the correction procedures, since the correction procedures generally lead to a better final result for input data with a small error than for input data with a large error. The iterative repetition is terminated when a convergence criterion is reached. Possible convergence criteria are: a) whether the change in projection geometry is less than a threshold; b) whether the change in the final volume is less than a threshold; c) whether the number of iteration steps is greater than a threshold; d) whether the computation time is greater than a threshold.

According to the present invention, the data sets generated by the above embodiments may be presented to a physician for visualization, in particular for diagnostic purposes, preferably by means of a display (9) or a printout.

In a preferred embodiment, the first correction method is performed as an image-to-image transformation, specifically in this case as a volume-to-volume transformation. The training of the ML network is performed with corresponding data pairs each consisting of a volume with motion artifacts and a volume without motion artifacts of the same recorded measurement object. The measurement object can be a human skull or technical test specimens of the dental DVT X-ray device or phantoms with anatomical tooth and skull structures. The data pairs may be generated by simulation. In one embodiment, the simulation of the volume with motion artifacts is performed by varying the reconstruction parameters of a DVT imaging without motion artifacts. In another embodiment, the simulation of the volume with motion artifacts is performed by generating simulated sinograms from a volume without motion artifacts while varying the projection parameters and then reconstructing the volume from the simulated sinogram. In another embodiment, the volume without motion artifacts is calculated using a conventional method for parameter estimation from a DVT imaging with motion artifacts. In yet another embodiment, the volumes with motion artifacts are generated by moving the target during the imaging. The movement of the target may be intentional or deliberate.

There are a variety of conventional methods for parameter estimation in the literature. In step S4, a method for parameter estimation is applied that calculates updated geometric parameters from a given volume and given measurement data. In a preferred embodiment, the estimation of the geometric parameters is performed by evaluating the differences between a forward projection of the given volume and the measured data. This corresponds to an evaluation of data fidelity, or a registration method. In a further embodiment, the estimation of the geometric parameters is performed as part of an iterative reconstruction method that takes into account prior knowledge or boundary conditions in a penalty term. Thus, the difference to the given volume and/or the difference to initially assumed geometric parameters may enter as a regularization. Herein, similarity measures are used, which, for example, compare the histograms, the gradients and/or the gray values of the volumes.

In step S5, a method for parameter estimation is applied that computes updated geometric parameters from given geometric parameters and given measurement data. In one embodiment, the estimation of the geometric parameters is performed by improving a metric for evaluating the image quality of the volume. In another embodiment, the estimation of the geometric parameters is performed using evaluation of data consistency constraints in the sinogram. In yet another embodiment, the estimation of the geometric parameters is performed using an iterative reconstruction method that iteratively reduces the difference between the forward projections of a reconstructed volume and the measured data. In a further embodiment, the geometric parameters are estimated using an iterative registration method that iteratively reconstructs a volume with the current geometric parameters and then estimates the geometric parameters by registering the measurement data with the reconstructed volume.

1. A method for geometric calibration of a DVT imaging by updating geometric parameters used in a reconstruction method, wherein the updating of the geometric parameters is supported by a first correction method based on machine learning (ML) by using the result of the first correction method as a reference for a second correction method for parameter estimation, and wherein the second correction method for parameter estimation involves measurement data of the DVT imaging, comprising:
(S1) providing the measurement data of the DVT imaging and the geometric parameters;
(S2) providing a first volume by applying a reconstruction method to the provided measurement data and the geometric parameters;
(S3) providing a corrected volume by applying the first correction method to the first volume;
(S4) providing updated geometric parameters by applying the second correction method to the measurement data and the corrected volume.

2. The method of claim 1, wherein the second correction method for parameter estimation of (S4) comprises a registration method that registers the measurement data with the corrected volume of (S3).

3. The method of claim 1, wherein the second correction method for parameter estimation from (S4) comprises an iterative reconstruction method using the corrected volume from (S3) for regularization.

4. The method according to claim 1, wherein the second correction method for parameter estimation of (S4) is performed only on a selected sub-region of the corrected volume and/or a selected sub-region of the measurement data, wherein the selection takes place by comparing the corrected volume and the first volume, or alternatively takes place directly by the first correction method.

5. A method according to claim 1, comprising:
(S6) providing a final corrected volume by applying a final reconstruction method to the measurement data and the updated geometric parameters from (S4).

6. A method according to claim 1, comprising,
(S5) providing re-updated geometric parameters by applying a third correction method for parameter estimation to the measured data and the updated geometric parameters from (S4);
(S6′) providing a final corrected volume by applying a final reconstruction method to the measured data and the re-updated geometric parameters from (S5).

7. The method of claim 5, wherein the final reconstruction method of (S6) generates sub-regions of the final volume, and combines them to form the final volume.

8. The method of claim 5, wherein the final reconstruction method of (S6) interpolates or extrapolates the updated geometric parameters of the sub-regions of (S4) or the re-updated geometric parameters of the sub-regions of (S5), and wherein the interpolation or extrapolation weights are determined from the relative location of the non-selected sub-regions in the volume to the selected sub-regions in the volume.

9. The method of claim 5, wherein the final reconstruction method of (S6) interpolates or extrapolates the updated geometric parameters of the sub-regions of (S4) or the re-updated geometric parameters of the sub-regions of (S5), and wherein the interpolation or extrapolation weights are determined from the relative spatial or temporal location of the non-selected sub-regions of the measurement data to the selected sub-regions of the measurement data.

10. The method according to claim 5, wherein (S3), (S4) and (S6), or (S3) to (S6) are repeated one or more times or are also performed iteratively.

11. A computer-assisted DVT system comprising computer-readable code which, when executed by a processor, causes the computer-assisted DVT system to:
(S1) provide measurement data of a DVT imaging and geometric parameters;
(S2) providing a first volume by applying a reconstruction method to the provided measurement data and the geometric parameters;
(S3) providing a corrected volume by applying a first correction method to the first volume;
(S4) providing updated geometric parameters by applying the second correction method to the measurement data and the corrected volume,
wherein an updating of the geometric parameters is supported by the first correction method based on machine learning (ML) by using the result of the first correction method as a reference for the second correction method for parameter estimation, and wherein the second correction method for parameter estimation involves measurement data of the DVT imaging.

12. A non-transitory computer readable storage medium storing a program, comprising instructions which when executed by a computer causes the computer to:
(S1) provide measurement data of a DVT imaging and geometric parameters;
(S2) providing a first volume by applying a reconstruction method to the provided measurement data and the geometric parameters;
(S3) providing a corrected volume by applying a first correction method to the first volume;
(S4) providing updated geometric parameters by applying the second correction method to the measurement data and the corrected volume,
wherein an updating of the geometric parameters is supported by the first correction method based on machine learning (ML) by using the result of the first correction method as a reference for the second correction method for parameter estimation, and wherein the second correction method for parameter estimation involves measurement data of the DVT imaging.

13. (canceled)

14. The method of claim 6, wherein the final reconstruction method of (S6′) generates sub-regions of the final volume, and combines them to form the final volume.

15. The method of claim 6, wherein the final reconstruction method of (S6′) interpolates or extrapolates the updated geometric parameters of the sub-regions of (S4) or the re-updated geometric parameters of the sub-regions of (S5), and wherein the interpolation or extrapolation weights are determined from the relative location of the non-selected sub-regions in the volume to the selected sub-regions in the volume.

16. The method of claim 6, wherein the final reconstruction method of (S6′) interpolates or extrapolates the updated geometric parameters of the sub-regions of (S4) or the re-updated geometric parameters of the sub-regions of (S5), and wherein the interpolation or extrapolation weights are determined from the relative spatial or temporal location of the non-selected sub-regions of the measurement data to the selected sub-regions of the measurement data.
